(19) United States
(12) Patent Application Publication
(10) Pub. No.: US 2007/0224156 A1
(43) Pub. Date: Sep. 27, 2007

(54) SHAMPOO FORMULATION FOR ANIMALS

(75) Inventor: Tony de Vos, Covina, CA (US)

Correspondence Address: DALE F. REGELMAN, LAW OFFICE OF DALE F. REGELMAN, P.C., 4231 SOUTH FREMONT AVENUE, TUCSON, AZ, 85714, US

(73) Assignee: CARDINAL LABORATORIES, INC., AZUSA, CA (US)

(21) Appl. No.: 11/689,475

(22) Filed: Mar. 21, 2007

Related U.S. Application Data
(60) Provisional application No. 60/784,342, filed on Mar. 21, 2006.

(51) Int. Cl. A61K 8/97

(52) U.S. Cl. 424/74; 424/777; 424/773; 424/778

(57) ABSTRACT

A shampoo for animals is disclosed. The shampoo formulation comprises Vitamin A and Vitamin E. In certain embodiments, the shampoo formulation further comprises one or more fruit extracts. In certain embodiments, those one or more fruit extracts are selected from the group consisting of Carica Papaya Fruit Extract, Psidiuim Guajava Fruit Extract, Passiflora Incamata Fruit Extract, Plumaria Acutifolia Flower Extract, Mangifera Indica Fruit Extract, and Colocasia Esculenta Root Extract.

### CROSS REFERENCE TO RELATED APPLICATIONS

This Application claims priority from a U.S. Utility Application having Ser. No. 60/784,342 filed Mar. 21, 2006, which is hereby incorporated herein.

### BACKGROUND OF THE INVENTION

All mammals have some sort of hair covering some, much, or virtually all of their bodies. The hair on some animals, particularly those traditionally trapped for their pelage, is known as fur, but it's all the same no matter the name.

Hair is made of the protein keratin and dead epidermal or skin cells and it grows from follicles in the dermis or inner layer of the skin. Some hairs are densely packed into stiff, fibrous outgrowths that, depending on species and location on the body, become horns, fingernails, and toenails.

Human follicles each give rise to a single hair, but animals that depend on fur coats for temperature regulation often have several or many hairs per follicle. The diameter of individual hairs decreases as the number of hairs per follicle increases.

Each follicle has an oil or sebaceous gland to keep skin and hair smooth and elastic. Dog breeds developed to work in water generally have a high oil content in their coat so that water runs of the guard hairs and does not penetrate to the skin.

Dogs have three types of hair: soft downy undercoat that is especially abundant in northern breeds but exists in most breeds that developed in cool or cold climates; stiffer and often longer guard hairs that form a protective layer to protect undercoat and skin from harsh weather and cold water; and whiskers, those specialized hairs that grow in clumps on the face.

Prior art shampoo formulations for animals suffered from unacceptably long drying times. Such elongated drying times limited the productivity of professional pet groomers. What is needed is an animal shampoo that exhibits decreased drying times, and that also enhances the structure and appearance of an animal's coat.

### SUMMARY OF THE INVENTION

The invention comprises a shampoo for animals. Applicant's shampoo formulation comprises Vitamin A and Vitamin E. In certain embodiments, the shampoo formulation further comprises one or more fruit extracts. In certain embodiments, those one or more fruit extracts are selected from the group consisting of Carica Papaya Fruit Extract, Psidiuim Guajava Fruit Extract, Passiflora Incamata Fruit Extract, Plumaria Acutifolia Flower Extract, Mangifera Indica Fruit Extract, and Colocasia Esculenta Root Extract.

### DETAILED DESCRIPTION OF THE PREFERRED EMBODIMENT

Reference throughout this Applicant to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. In the following description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

Applicant's invention includes an animal shampoo formulation. In certain embodiments, Applicant's shampoo formulation is formulated for use with companion animals, such as dogs and cats. Table I summarizes the components and weight percentages of those components comprising the various embodiments of Applicant's formulation. In addition to the other benefits resulting from use of Applicant's shampoo formulation, use of Applicant's shampoos formulation gives a reduced drying time compared to prior art pet shampoos. Such a reduced drying time is of great benefit to professional pet groomers. Drying times realized using Applicant's pet shampoo formulation are between about 18% to about 25% less than drying times using prior art pet shampoo formulations.

Applicant has discovered that the drying time using his shampoo formulation is a function of ambient environmental conditions, such as and without limitation temperature and humidity, and also the characteristics of the pet's coat, such as and without limitation length of hair fibers, coat packing density, and the like.

TABLE I

| COMPONENT | WEIGHT PERCENT |
|---|---|
| Water | 87–97 |
| Vitamin A | 0–1 |
| Vitamin E | 0–1 |
| Fruit Extracts | 0–1 |
| Tannins | 0–1 |
| Gallic Acid | 0–1 |
| Catechins | 0–1 |
| Proanthocyanins | 0–1 |
| Flavonoids | 0–1 |
| Essential Oils | 0–1 |
| Choline | 0–1 |
| Spanonins | 0–1 |
| Protein | 0–1 |
| Silk amino acids | 0–1 |

Table II recites a plurality of fruit extracts used in the various embodiments of Applicant's shampoo formulation. Each embodiment of Applicant's formulation comprises at least one of the fruit extracts recited in Table II. Certain embodiments of Applicant's formulation comprise all of the fruit extracts recited in Table II.

TABLE II

| FRUIT EXTRACT | FUNCTION |
|---|---|
| Carica Papaya Fruit Extract | Exfoliates and refines surface |
| | textures for a smoother, cleaner look |
| Psidiuim Guajava Fruit Extract | Source of Vitamin C. Stimulates |
| | collagen tythesis |
| Passiflora Incamata Fruit Extract | Source of Vitamin A, Vitamin C, and |
| | Potassium |
| Plumaria Acutifolia Flower Extract | Fragrance |
| Mangifera Indica Fruit Extract | Improves elasticity in hair and skin |
| Colocasia Esculenta Root Extract | Anti irritant |

Applicant has found that the Vitamin A component promotes collagen growth, and improves skin elasticity. By “Vitamin A,” Applicant mean Vitamin A precursors, including one or more β-carotene, and one or more of four biologically active molecules, including, all-trans retinal (Compound I), 11-cis-retinal (Compound II), retinol (Compound III), and retinoic acid, (Compound IV).

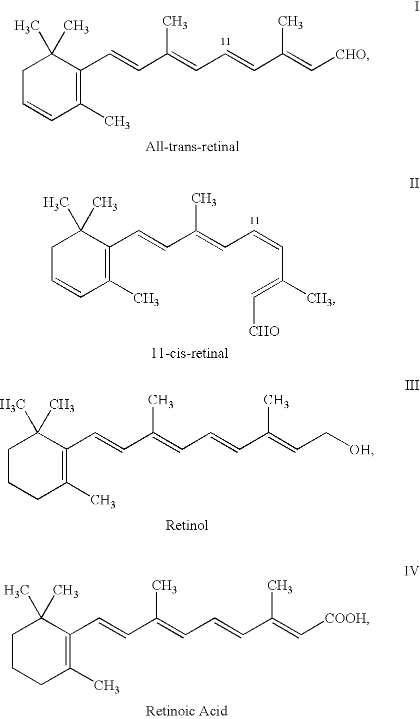

Each of these compounds are derived from the plant precursor molecule, β-carotene (a member of a family of molecules known as carotenoids). Beta-carotene, which consists of two molecules of retinal linked at their aldehyde ends, is also referred to as the provitamin form of vitamin A.

Applicant has found that Vitamin E functions as an anti exident nutrient, and also provides U.V. protection to the animal's coat. By “Vitamin E,” Applicant mean a mixture of several related compounds known as tocopherols. The α-tocopherol molecule V is the most potent of the tocopherols.

The major function of vitamin E is to act as a natural antioxidant by scavenging free radicals and molecular oxygen. In particular vitamin E is important for preventing peroxidation of polyunsaturated membrane fatty acids. The vitamins E and C are interrelated in their antioxidant capabilities. Active α-tocopherol can be regenerated by interaction with Vitamin C following scavenge of a peroxy free radical. Alternatively, α-tocopherol can scavenge two peroxy free radicals and then be conjugated to glucuronate for excretion in the bile.

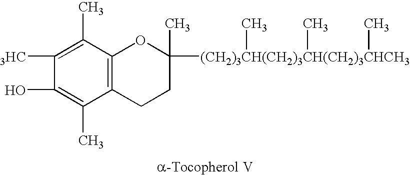

Tannins comprise astringent, bitter-tasting plant polyphenols that bind and precipitate proteins. The term tannin refers to the source of tannins used in tanning animal hides into leather; however, the term is applied to any large polyphenolic compound containing sufficient hydroxyls and other suitable groups (such as carboxyls) to form strong complexes with proteins and other macromolecules. Tannins have molecular weights ranging from 500 to over 20,000.

Tannins are usually divided into hydrolyzable tannins and condensed tannins (proanthocyanidins). At the center of a hydrolyzable tannin molecule, there is a polyol carbohydrate (usually D-glucose). The hydroxyl groups of the carbohydrate are partially or totally esterified with phenolic groups such as gallic acid (in gallotannins) or ellagic acid (in ellagitannins). Hydrolyzable tannins are hydrolyzed by weak acids or weak bases to produce carbohydrate and phenolic acids. Condensed tannins, also known as proanthocyanidins, are polymers of 2 to 50 (or more) flavonoid units that are joined by carbon-carbon bonds, which are not susceptible to being cleaved by hydrolysis. While hydrolyzable tannins and most condensed tannins are water soluble, some very large condensed tannins are insoluble.

Examples of gallotannins are the esters of tannic acid (C₇₆H₅₂O₄₆) with glucose, found in the leaves and bark of many plant species. Gallic acid, compound VI, comprises an organic acid, also known as 3,4,5-trihydroxybenzoic acid, found in gallnuts, sumac, witch hazel, tea leaves, and other plants. The chemical formula is C₆H₂(OH)₃CO₂H. Gallic acid is found both free and as part of tannins. The COOH moeity has a pKa of 4.5, and the phenolic hydroxyl moeities have a pKa of 10.

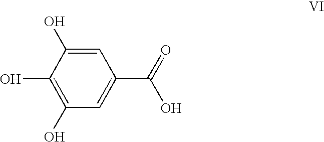

Catechins, including Epicatechin VII and Epigallocatechin VIII, comprise bioflavonoids, polyphenols and powerful anti-oxidants. Catechins are believed to have some value in fighting tumors as well as enhancing immune system function. Catechin is a powerful, water soluble polyphenol and antioxidant that is easily oxidized.

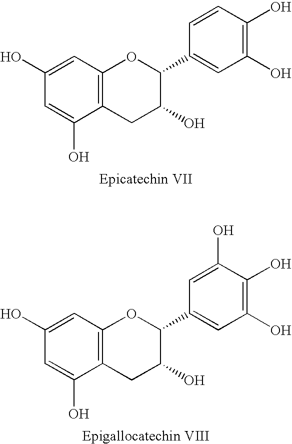

Anthocyanins comprise powerful antioxidants, and belong to the class of flavonoids. The anthocyanins themselves are subdivided into the sugar-free anthocyanidine aglycons and the anthocyanin glycosides. They are considered secondary metabolites and allowed as a food additive with E number 163. Approximately 250 different anthocyanins are known.

As a general matter, flavonoids comprise a class of plant secondary metabolites based around a phenylbenzopyrone structure IX. Flavonoids are most commonly known for their antioxidant activity. Flavonoids are also commonly referred to as bioflavonoids in the media—these terms are equivalent and interchangable, since all flavonoids are biological in origin.

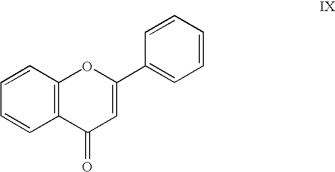

Kaempferol, compound X comprising 3,5,7-trihydroxy-2-(4-hydroxyphenyl)-4H-1-benzopyran-4-one, comprises is a natural flavonoid which has been isolated from Delphinium, Witch-hazel, and other plant sources. Kaempferol is a yellow crystalline solid with a melting point of 276-278° C. It is slightly soluble in water, and well soluble in hot ethanol and diethyl ether. Many glycosides of kaempferol, such as kaemferitrin and astragalin, have been isolated as natural products from plants.

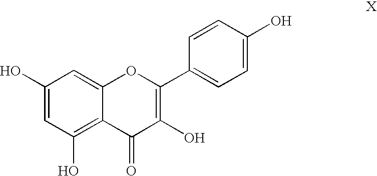

An essential oil comprises a concentrated, hydrophobic liquid containing volatile aromatic compounds extracted from plants. Essential oil is also known as volatile oil and ethereal oil. Such materials are sometimes referred to as “oil of” the raw plant material from which it was extracted, such as oil of clove. The term essential is intended to convey that the oil is an essence of the plant it is extracted from, and not in the more common sense of being indispensible.

Carvacrol, or cymophenol, compound XI—C₆H₃CH₃(OH)(C₃H₇), is a constituent of the ethereal oil of Origanum hirtum, oil of thyme, oil obtained from pepperwort, and wild bergamot. It may be synthetically prepared by the fusion of cymol sulphonic acid with caustic potash; by the action of nitrous acid on 1-methyl-2-amino-4-propyl benzene; by prolonged heating of five parts of camphor with one part of iodine; or by heating carvol with glacial phosphoric acid or by preforming a dehydrogenation of carvone with a Pd/C catalyst. It is extracted from Origanum oil by means of a 50% potash solution. It is a thick oil which sets at 20° C. to a mass of crystals of melting point 0° C., and boiling point 236-237° C. Oxidation with ferric chloride converts it into dicarvacrol, whilst phosphorus pentachloride transforms it into chlorcymol.

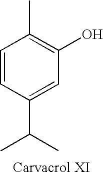

Eugenol, compound XII—C₁₀H₁₂O₂, is an allyl chain-substituted guaiacol, i.e. 2-methoxy-4-(2-propenyl)phenol. It is a clear to pale yellow oily liquid extracted from certain essential oils especially from clove oil and cinnamon. It's slightly soluble in water and soluble in organic solvents. It has a pleasant, spicy, clove-like odor. Eugenol is used in perfumeries, flavorings, essential oils and in medicine as a local antiseptic and anaesthetic. Eugenol derivatives or methoxyphenol derivatives are used in formulating analgesics, biocides and antiseptics.

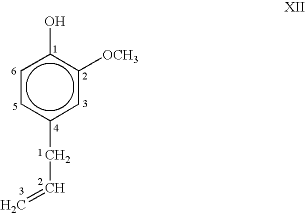

Cis-3-Hexen-1-ol, compound XIII and also known as (Z)-3-hexen-1-ol and leaf alcohol, is a colorless oily liquid with an intense grassy-green odor of freshly cut green grass and leaves.

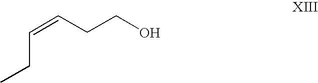

Choline is a quaternary saturated amine with the chemical formula (CH₃)₃N⁺CH₂CH₂OHX⁻ wherein X⁻ is a counterion such as chloride, hydroxide, and/or tartrate. Choline is a nutrient, essential for cardiovascular and brain function, and for cellular membrane composition and repair. Choline and its metabolites are needed for three main physiological purposes: structural integrity and signaling roles for cell membranes, cholinergic neurotransmission (acetylcholine synthesis), and as a major source for methyl-groups via its metabolite, trimethylglycine that participates in the S-adenosylmethionine synthesis pathways.

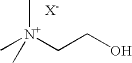

Saponins are glycosides of steroids, steroid alkaloids (steroids with a nitrogen function) or triterpenes found in plants, especially in the plant skins where they form a waxy protective coating. They dissolve in water to form a soapy froth.

In certain embodiments, Applicant's shampoo formulation comprises Wheat Proteins, wherein those Wheat Proteins comprise moisturizers and/or humectants. In certain embodiments, Applicant's shampoo formulation comprises one or more silk amino acids, wherein those one or more silk amino acids comprise moisturizers, and promoters of Collagen.

In certain embodiments, Applicant's shampoo formulation is formed using an extract of the leaves of the witch-hazel plant Hamamelidaceae Hamamelis virginiana L. In certain embodiments, Applicant's shampoo formulation is formed using a distillate of dried plant materials, including twigs, from the witch-hazel plant.

While the preferred embodiments of the present invention have been illustrated in detail, it should be apparent that modifications and adaptations to those embodiments may occur to one skilled in the art without departing from the scope of the present invention as defined by the claims set forth below.

I claim:

1. A shampoo for animals, comprising:
Vitamin A; and
Vitamin E.

2. The shampoo of claim 1, further comprising one or more tannins.

3. The shampoo of claim 2, further comprising Gallic acid.

4. The shampoo of claim 3, further comprising choline.

5. The shampoo of claim 4, further comprising Epicatechin and Epigallocatechin.

6. The shampoo of claim 5, further comprising one or more flavonoids.

7. The shampoo of claim 6, further comprising Kaempferol.

8. The shampoo of claim 6, further comprising one or more essential oils.

9. The shampoo of claim 8, further comprising Carvacrol.

10. The shampoo of claim 8, further comprising Eugenol.

11. The shampoo of claim 10, further comprising (Z)-3-hexen-1-ol.

12. The shampoo of claim 1, further comprising one or more fruit extracts.

13. The shampoo of claim 12, wherein said one or more fruit extracts are selected from the group consisting of Carica Papaya Fruit Extract, Psidiuim Guajava Fruit Extract, Passiflora Incamata Fruit Extract, Plumaria Acutifolia Flower Extract, Mangifera Indica Fruit Extract, and Colocasia Esculenta Root Extract.
